(19) United States
(12) Patent Application Publication
(10) Pub. No.: US 2016/0324628 A1
(43) Pub. Date: Nov. 10, 2016

(54) ELECTRO-OPTICAL MONOFOCAL INTRAOCULAR LENS

(71) Applicants: Amitava GUPTA, Roanoke, VA (US); Rudy MAZZOCCHI, Roanoke, VA (US); Roel TRIP, Roanoke, VA (US); Urban SCHNELL, Roanoke, VA (US); Jean-Noel FEHR, Roanoke, VA (US); ELENZA, INC., Roanoke, VA (US)

(72) Inventors: Amitava GUPTA, Roanoke, VA (US); Rudy MAZZOCCHI, Rockledge, FL (US); Roel TRIP, Suwanee, GA (US); Urban SCHNELL, Munchenbuchsee (CH); Jean-Noel FEHR, Neuchatel (CH)

(73) Assignee: Elenza ,Inc., Roanoke, VA (US)

(21) Appl. No.: 15/110,329

(22) PCT Filed: Jan. 7, 2015
(86) PCT No.: PCT/US2015/010489

§ 371 Date: Jul. 7, 2016

Related U.S. Application Data
(60) Provisional application No. 61/925,092, filed on Jan. 8, 2014.

(51) Int. Cl. A61F 2/16; A61B 5/00

(52) U.S. Cl. CPC A61F 2/1624; A61B 5/0059; A61F 2/1659; A61F 2002/1696

(57) ABSTRACT

Described herein is an implantable intraocular lens that can automatically adjust its optical power based on the eye's natural response for accommodation of targets at varying distances. The implantable intraocular lens includes a physiological sensor for detecting a physiological response of an eye associated with an ocular accommodation, and an electro-optical element configured to adjust optical power based on the detected physiological response of the eye.

### CROSS REFERENCE TO RELATED APPLICATION

This application claims the benefit of U.S. Provisional Application Ser. No. 61/925,092 filed on Jan. 8, 2014 and entitled “An Intraocular Lens Implant Comprising An Electrically Switchable Optical Element,” the contents of which are incorporated herein by reference in their entirety.

### FIELD OF THE DISCLOSURE

The present disclosure, in general, relates to an implantable electro-active intraocular lens for vision correction.

### BACKGROUND

Presbyopia is the loss of accommodation of the crystalline lens of the human eye that often accompanies aging. Presbyopia often results in the eye of a person being unable to focus on near distance objects and later unable to focus on intermediate distance objects. It is estimated that there are approximately 90 million to 100 million presbyopes in the United States, and approximately 1.6 billion presbyopes worldwide.

The standard tools for correcting presbyopia include reading glasses, multifocal ophthalmic lenses, and contact lenses fit to provide monovision. Reading glasses usually have a single optical power for correcting near distance focusing problems. Contact lenses fit to provide monovision are two contact lenses having different optical powers: one contact lens for correcting mostly far distance focusing problems and the other contact lens for correcting mostly near distance focusing problems. These glasses are not capable of mimicking or replacing the eye's natural accommodation response.

A multifocal lens has more than one focal length (i.e., optical power) for correcting focusing problems across a range of distances. Multifocal optics are used in eyeglasses, contact lenses, and intraocular lenses. Multifocal ophthalmic lenses include divided regions of different optical powers. Multifocal lenses may comprise continuous surfaces that create continuous optical power, such as in a progressive lens (e.g., progressive addition lens (PAL)). However, progressive lenses create regions of aberration away from the optic axis, yielding poor visual resolution (blur). There is also an inherent impact on peripheral vision with progressive lenses which is more obvious than that found in single vision lenses. Therefore, some wearers find the visual discomfort caused by these distortions outweigh the benefits of wearing PALs. In addition, progressive lenses require careful placement relative to the wearer's pupil center for a distance-viewing reference position. Alternative to progressive lenses, multifocal lenses may comprise discontinuous surfaces that create discontinuous optical power, such as in bifocals or trifocals. Bifocals or trifocals can cause headaches and even dizziness in some users. Additionally, acclimation to the small field of view offered by the reading segment of bifocals or trifocals may be difficult for some users.

Conventional intraocular lenses are monofocal, spherical lenses that provide focused retinal images for far objects. Generally, the focal length (or optical power) of a spherical intraocular lens is chosen based on viewing a far object that subtends a small angle (e.g., about seven degrees) at the fovea. Because monofocal intraocular lenses have a fixed focal length, they are not capable of mimicking or replacing the eye's natural accommodation response.

It would be beneficial to have available ophthalmic devices which mimic or replace the eye's natural accommodation response.

### SUMMARY

The present disclosure provides, in some embodiments, an electronic intraocular lens comprising: a physiological sensor for detecting a physiological response of an eye associated with an ocular accommodation; and an electro-optical element configured to adjust optical power based on the detected physiological response of the eye.

In some embodiments, the physiological sensor of the electronic intraocular lens is configured to measure the illumination level at a plane where the electronic intraocular lens is implanted. In some embodiments, the physiological sensor of the electronic intraocular lens is a photovoltaic cell.

In some embodiments, the electro-optical element of the electronic intraocular lens is a lens comprising transparent electrodes, and electro-optical material filled in a cavity formed by a transparent material. In such embodiments, the lens is one of a diffractive lens and a refractive lens. In some embodiments, the electro-optical element of the electronic intraocular lens is capable of adjusting optical power continuously or in multiple discrete levels.

In some embodiments, the electronic intraocular lens further comprises a logic controller electrically connected to the physiological sensor and the electro-optical element, wherein the logic controller is configured to analyze the physiological response measured by the physiological sensor, determine an appropriate adjustment of the optical power based on the measured physiological response, and control the electro-optical element to achieve the appropriate adjustment of the optical power.

In some embodiments, the electronic intraocular lens further comprises at least one non-volatile memory electrically connected to the logic controller, wherein the non-volatile memory stores codes to be executed by the logic controller to configure and control the electronic intraocular lens.

In some embodiments, the electronic intraocular lens further comprises an ambient light sensor configured to measure the ambient light level, wherein the logic controller analyzes the measured physiological response together with the measured ambient light level to determine the appropriate adjustment of the optical power.

In some embodiments, the electronic intraocular lens further comprises: at least one rechargeable battery; and at least one of an antenna and a photovoltaic cell for charging the rechargeable battery. In some embodiments, the rechargeable battery comprises a solid-state thin-film battery.

In some embodiments, the electronic intraocular lens further comprises a rechargeable battery, and an antenna including an inductive coil for receiving energy to recharge the battery. In some embodiments, the antenna can be configured for wireless data communication in addition to wirelessly charging the rechargeable battery. In some embodiments, the electronic intraocular lens can be remotely switched off through the antenna or manually switched off by specific predefined movement of the eye.

In some embodiments, the electronic intraocular lens further comprises a power management circuit for managing the charging of the rechargeable battery and distributing power to other components in the electronic intraocular lens.

In some embodiments, the physiological sensor and the electro-optical element of the electronic intraocular lens are encapsulated or hermetically sealed by at least one biocompatible material.

The present disclosure also provides, in some embodiments, a monofocal intraocular lens comprising: a photovoltaic sensor for sensing pupil constriction and dilation; an electronic circuit electrically connected to the photovoltaic sensor; and an electrically switchable optical element electrically connected to the electronic circuit; wherein the electronic circuit analyzes a signal from the photovoltaic sensor and generates a control signal to cause a selected control voltage or current level to be applied to the electrically adjustable optical element, thereby modifying an optical power of the electrically switchable optical element. In some embodiments, the selected control voltage or current level switches the electrically adjustable optical element on or off, and the optical power of the electrically adjustable optical element is zero when switched off.

The present disclosure further provides, in some embodiments, a method of artificial ocular accommodation comprising: detecting, by a physiological sensor, a physiological response of an eye of a person associated with a natural ocular accommodation; and adjusting an optical power of an electro-optical element based on the detected physiological response of the eye.

In some embodiments, the method of artificial ocular accommodation further comprises at least one of filtering noise of the detected physiological response and customizing the adjustment of the optical power of the electro-optical element based on the person's pupil dynamics.

Other aspects and embodiments of the disclosure are also contemplated. The foregoing summary and the following detailed description are not meant to restrict the disclosure to any particular embodiment but are merely meant to describe some embodiments of the disclosure.

### BRIEF DESCRIPTION OF DRAWINGS

FIGS. 1A-1D illustrate examples of physiological response of an eye associated with ocular accommodation. FIG. 1A is the side view of an eye in far vision. FIG. 1B is the front view of an eye in far vision. FIG. 1C is the side view of an eye in near vision. FIG. 1D is the front view of an eye in near vision.

FIG. 2 illustrates an example of an electronic intraocular lens as positioned when implanted in the eye of a patient.

FIG. 3 illustrates an embodiment of an electro-optical lens including an electro-optical element in the form of a diffractive lens.

FIG. 4 provides an exploded view of an embodiment of an electronic intraocular lens.

FIG. 5 illustrates an embodiment of an electronic intraocular lens.

FIG. 6 provides an overview of functional blocks of an embodiment of an electronic intraocular lens.

FIGS. 7A-7D illustrate different ways of wirelessly charging a rechargeable battery in the intraocular lens using different external devices, such as a sleeping eye mask in FIG. 9A; a sleeping pillow in FIG. 9B; goggles in FIG. 9C; and a contour neck pillow in FIG. 9D.

FIG. 8A provides an embodiment of a haptic design of an implantable intraocular lens.

FIG. 8B provides another embodiment of a haptic design of an implantable intraocular lens.

FIG. 9 illustrates an embodiment of an intraocular lens inserter and a use of the inserter to implant an intraocular lens.

FIG. 10 illustrates a technique for initializing and customizing an implanted electronic intraocular lens.

FIG. 11 illustrates a technique for artificial accommodation using an electronic intraocular lens in a normal functional mode.

FIG. 12 illustrates some embodiments of conditions that can trigger switching off of the electro-optical element in the intraocular lens.

Some or all of the figures are schematic representations by way of example; hence, they do not necessarily depict the actual relative sizes, locations, or sequences of the elements shown. The figures are presented for the purpose of illustrating one or more embodiments with the explicit understanding that they will not be used to limit the scope or the meaning of the claims that follow below.

### DETAILED DESCRIPTION

Ophthalmic devices with electro-active elements that can monitor pupil dynamics associated with natural accommodation, and provide variable optical power accordingly to mimic or replace the eye's natural accommodation response, are described in this disclosure. Natural accommodation indicates the refocusing of the eye lens based on a target focus distance.

The following examples and embodiments serve to illustrate the present disclosure. These examples are in no way intended to limit the scope of the disclosure.

Implantable ophthalmic devices, such as intraocular lenses, intraocular implants, corneal inlays, and corneal onlays, are typically implanted in the eye to serve as permanent or quasi-permanent correction for presbyopia, pseudophakia, aphakia, and other conditions affecting a patient's vision. Implantable ophthalmic devices may be inserted or implanted in the anterior chamber or posterior chamber of the eye, into the capsular sac or the stroma of the cornea (similar to a corneal inlay), into the epithelial layer of the cornea (similar to a corneal onlay), or within any anatomical structure of the eye. As described in this disclosure, an implantable ophthalmic device may be used to mimic or replace the eye's natural accommodation response: physiological responses of the eye normally associated with natural accommodation are measured, and optical power of the implantable ophthalmic device is accordingly adjusted to replace the accommodation response that no longer naturally occurs. One such physiological response that may be measured is pupillary response.

Pupillary response varies the size of the pupil via the optic and oculomotor cranial nerve. This response results in either constriction (miosis) that narrows the pupil, or dilation (mydriasis) that widens the pupil. Pupillary response occurs through relaxation or contraction of the iris dilator muscle. A pupil dilates and constricts in response to light levels and distance of the target object.

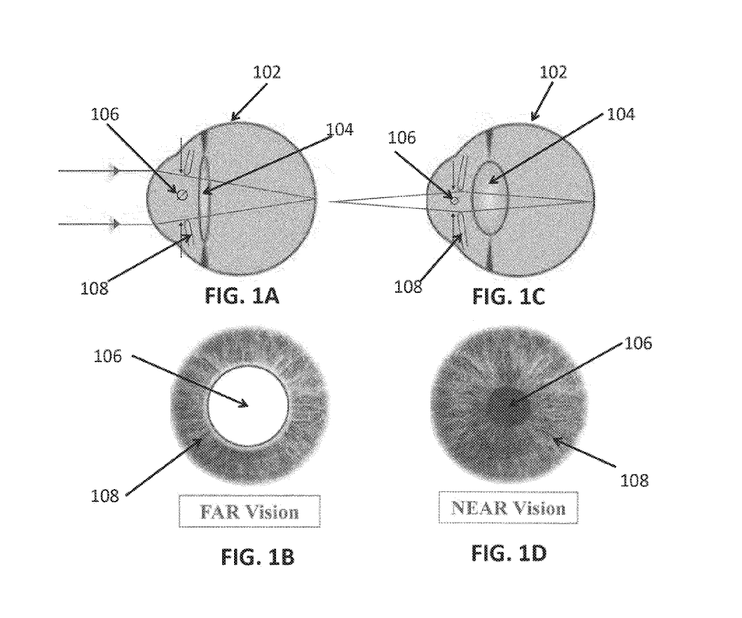

FIGS. 1A-1D illustrate the normal physiological responses (including pupillary response) of an eye associated with a natural accommodation. During distance viewing (FIGS. 1A and 1B), the iris dilator muscle 108 of an eye 102 relaxes, the pupil 106 dilates, and the crystalline lens 104 has a shorter depth and therefore a longer focal length or a lower optical power. During near viewing (FIGS. 1C and 1D), the iris dilator muscle 108 contracts, the pupil 106 constricts, and the crystalline lens 104 has a longer depth and therefore a shorter focal length and a higher optical power. The amount of light passing through the pupil 106 is limited by the aperture of the pupil. As depicted in FIGS. 1B and 1D, respectively, when the pupil 106 dilates, the aperture of the pupil 106 is larger, thus allowing more light to pass through; and when the pupil 106 constricts, the aperture of the pupil 106 is smaller, thus allowing less light to pass through.

The amount of light passing through the pupil 106 for different viewing distances may be characterized to identify a relationship between the pupillary response and the target focal distance. For example, for each of several target focal distances, the amount of light passing through a pupil may be measured, to create a characterization of the relationship between light measured and target focal distance. Such a characterization may be performed on multiple subjects to characterize the light versus focal distance relationship for a segment of, or all of, the population. A population segment may be a segment based on age, sex, ethnicity, eye color, or other feature. The more subjects used to create a characterization, the more applicable the characterization will generally be across the corresponding population segment. A complex characterization may include characterizations for multiple population segments.

A characterization may be used as a default, which default may subsequently be calibrated to compensate for a particular eye of a particular person, prior to or following implantation of an intraocular lens. In this manner, by calibrating a predefined default characterization, less time may be required to configure the intraocular lens for the particular patient. In some embodiments, the calibrated characterization may be stored as a baseline characterization for later reference, such as to track deterioration of a pupillary response.

Alternatively to characterization based on multiple subjects, a characterization may directly be made for an eye of a particular person to be implanted with an intraocular lens.

Once an intraocular lens according to this disclosure is implanted, the amount of light passing through the pupil 106 may be measured continuously or at intervals. In some embodiments, the measured amount of light is used to determine, based on the default or calibrated characterization, a corresponding target focal distance, and the determined target focal distance is then used to identify an amount by which the optical power of the implanted lens should be modified. In other embodiments, the measured amount of light is used to directly determine the amount by which the optical power of the implanted lens should be adjusted, or used to directly determine a control signal to apply to adjust the optical power of the implanted lens.

In some embodiments, the characterization (default or calibrated) is stored as a look-up table, in which the measured amount of light is used to determine an index into the look-up table to identify a corresponding target focal distance, a control signal to apply, or a desired optical power modification. In other embodiments, the characterization is in the form of an equation in which the measured amount of light is a variable. These embodiments are not limiting, and other embodiments of the characterization are also possible, such as the use of thresholds or models.

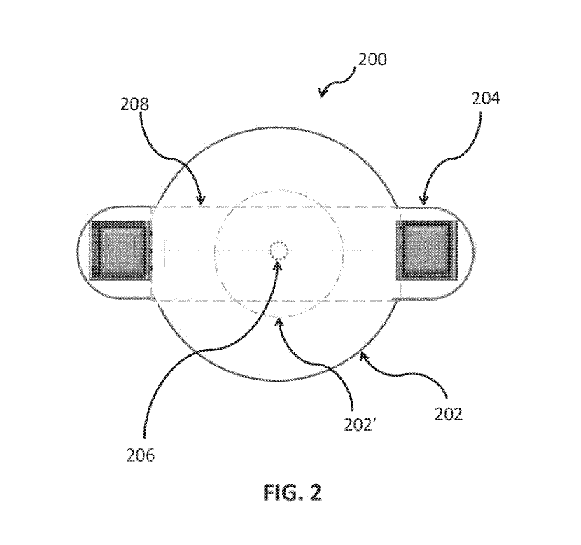

FIG. 2 illustrates a portion of an eye 200, showing positioning of an implanted electronic intraocular lens 204, in which full pupil dilation 202 is shown for far target focal distance, with an indication in dotted line of partial pupil dilation 202′ for nearer target focal distance. Electronic intraocular lens 204 includes a physiological sensor 206 for detecting a physiological response of the eye 200 as the target focal distance of the eye 200 changes. An electro-optical element 208 has variable optical power, which may be varied as the target focal distance changes, to mimic the eye's natural accommodation response to a change in target focal distance.

In some embodiments, the physiological sensor 206 of the implantable electronic intraocular lens 204 comprises a photovoltaic cell disposed behind the pupil of the eye 200. The photovoltaic cell can detect the amount of light passing through the pupil and reaching the plane where the intraocular lens 204 is implanted. From the amount of light detected, a desired focal power is determined based on the prior characterization (or calibrated characterization) described above. The electro-optical element 208 may then be controlled to modify the optical power of the electro-optical element 208.

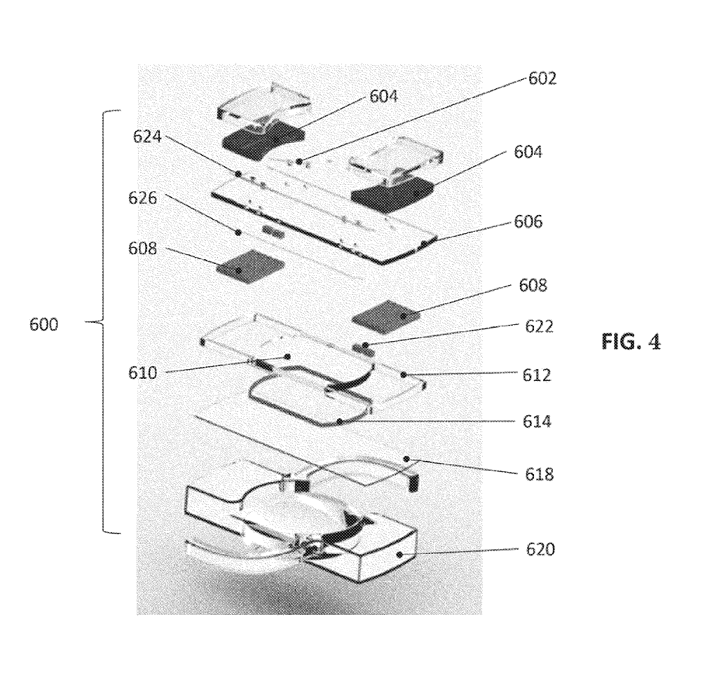

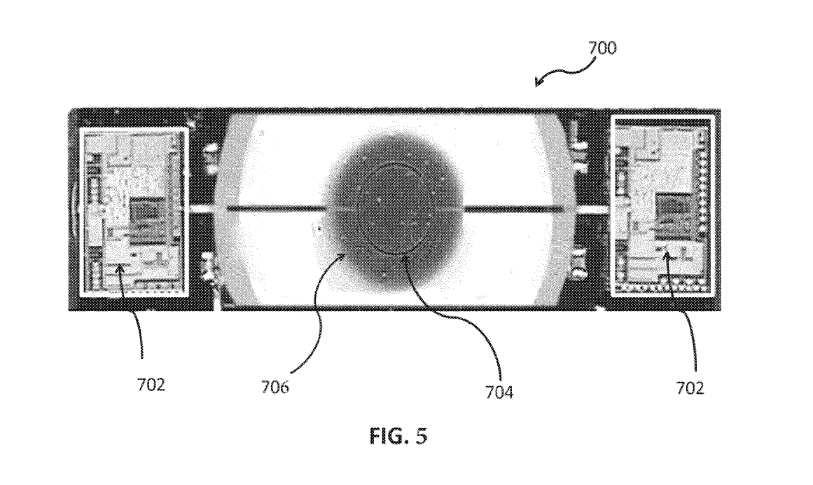

In some embodiments, because a pupil also dilates and constricts in response to changes in ambient light level, the modification of the optical power of the electro-optical element 208 takes into account the ambient light level. For example, an eye's natural response to ambient light may be characterized by measuring the pupillary dilation of one or more subjects at different ambient light levels. The characterization of pupillary dilation may then be used in an implanted intraocular lens 204 as an offset for the pupillary dilation due to a change in focal target. For example, if a look-up table is used to identify a target focal distance, a control signal to apply, or a desired optical power modification by determining an index into the table based on total measured light level, the look-up table may include an additional dimension related to ambient light level, and a measured ambient light level used as the basis for an additional index into the look-up table. For another example, if an equation is used in which measured light level is a variable, another variable in the equation could be related to measured ambient light level. For a further example, measured ambient light level may be subtracted from measured total light level for an adjusted light level used to determine target focal distance, a control signal to apply, or a desired optical power modification. In an embodiment in which the ambient light level is measured, the ambient light sensor is positioned outside of the pupillary area. Such an ambient light sensor may be external or internal to the intraocular lens 204. For example, the ambient light sensor may be a photosensor positioned at a periphery of the electro-optical element 208, or within a portion of the intraocular lens 204 containing electronic circuits (such as electronic circuits described below with respect to FIGS. 4 and 5).

The electro-optical element 208 of the intraocular lens 204 includes an electro-optical material, which changes properties in response to an electric field developed across the electro-optical material. One example of an electro-optic material is a liquid crystal. When a control voltage or current is applied to the electro-optical element 208, an electric field is caused to be developed across the electro-optic material, which effects a change in material properties. The change in material properties provides a change in optical power of the electro-optic material. Thus, optical power may be controlled by the control voltage or current applied to the electro-optical element 208.

The relationship of the applied control voltage or current to the resulting optical power, or the relationship of the change in applied control voltage or current to the resulting change in optical power, is pre-determined (e.g., through characterization and calibration), so that an appropriate control voltage or current to apply to the electro-optical element 208 to effectuate a desired optical power adjustment may be determined while the intraocular lens 204 is in use.

In some embodiments, the electro-optical element 208 may have a positive optical power at its default power-off state. In other embodiments, the electro-optical element 208 may have zero optical power at its default power-off state. In yet other embodiments, the electro-optical element 208 may have a negative optical power at its default power-off state.

An electro-optical material in the electro-optical lens 204 can be a material the refractive index of which reduces when the electric field increases, such that the optical power of the electro-optical element 208 reduces when the control voltage or current applied increases. Alternatively, the electro-optical material in the electro-optical lens 204 can be a material the refractive index of which increases when the electric field increases, such that the optical power of the electro-optical element 208 increases when the control voltage or current applied increases. In some embodiments, the electro-optical material in the electro-optical element 208 has a composition such that its default refractive index with no electrical field developed across the electro-optical material is matched to the refractive index of an optical structure that encloses the electro-optical material. In some embodiments, the default refractive index of the electro-optical material is matched to the refractive index of the fluid of the eye where the intraocular lens 200 is implanted, such as the vitreous humour.

The electro-optical lens 204 may include one or more lenses within, or on a surface of, electro-optical lens 204. A lens within or on a surface of electro-optical lens 204 may be refractive or diffractive. A refractive lens may be constructed, for example, by forming the lens with a concave or convex surface. A diffractive lens may be constructed by forming the lens with a patterned surface. A diffractive optical lens can achieve the same focal length as a refractive lens while using a material of much less thickness than that used in a refractive lens. Additionally, as compared with a refractive lens, a thinner diffractive electro-optical lens requires less power to develop the same electric field to effect a refractive index change in an electro-optical material for a given optical power. An example of a diffractive lens is provided next, illustrating one construction for implementation of a diffractive pattern.

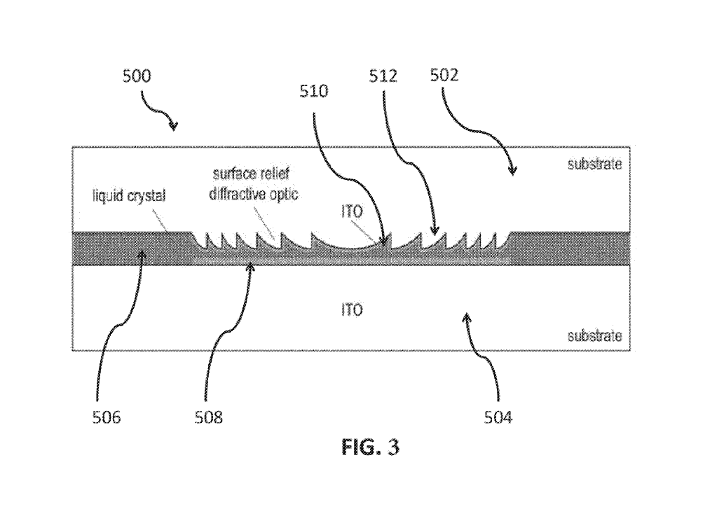

FIG. 3 depicts an embodiment of a diffractive-type electro-optical element 500. The electro-optical element 500 includes a top substrate 502 with a surface relief diffractive optical lens 512 formed on a bottom surface of the top substrate 502, a bottom substrate 504, and a cavity 506 defined between the top substrate 502 and the bottom substrate 504, the cavity filled with an electro-optical material (shown as a liquid crystal, although other electro-optical materials may be used instead). By way of example, the diffractive optical lens 512 on the top substrate 502 can be cast from a tooling replicated from a master. The electro-optical element 500 further includes two transparent electrodes 508 and 510 for applying a control voltage or current on the electro-optical element 500 to effect an electric field across the liquid crystal. A first transparent electrode 508 is deposited on a top surface of the bottom substrate 504. A second transparent electrode 510 is deposited on the bottom surface of the top substrate 502. The first transparent electrode 508 and the second transparent electrode 510 each may be formed, for example, as a thin film, using materials such as indium tin oxide.

FIG. 4 is an exploded view of an embodiment of an intraocular lens 600 according to this disclosure. The intraocular lens 600 includes a photovoltaic cell 602, rechargeable batteries 604, a top wafer 606, electronic circuits 608, an intermediate wafer 612, an RF coil 614, and a bottom wafer 618. At least portions of the wafers 606, 612, and 618 are formed of transparent material. A cavity 610 is defined by sealing apertures in the intermediate wafer 612 between the bottom wafer 618 and the top wafer 606, which can be bonded together using at least one of laser fusion bonding, pressure bonding, and anodic bonding. The electronic circuits 608 and additional electronic components, such as a capacitor 622, can be disposed within cavities defined in one or more of the top wafer 606, the intermediate wafer 612, and the bottom wafer 618. The electronic circuits 608 can be implemented, for example, as components on a printed circuit board. Other elements, such as the RF coil 614, the photovoltaic cell 602, and electrical lines 624 and 626 can also be affixed to, or positioned between, the wafers 606, 612, and 618. The cavity 610 may be filled with an electro-optical material. One or both of the top wafer 608 and the bottom wafer 618 may be constructed as a lens with a defined optical power, and the lens may be refractive or diffractive. For example, a substrate such as the diffractive substrate 502 illustrated in FIG. 3 may be used for the top wafer 608 or the bottom wafer 618 in the embodiment illustrated in FIG. 4. The intraocular lens 600 may be encapsulated entirely or partially by biocompatible materials 620, such as acryl.

FIG. 5 is a top view of one embodiment of an implanted intraocular lens 700, such as may be constructed from the components illustrated in FIG. 4. The implantable intraocular lens 700 includes electronic circuits 702. The electronic circuits may be located on one or more printed circuit boards, as shown, and hermetically sealed within cavities of the intraocular lens 700. In the embodiment illustrated in FIG. 5, the intraocular lens 700 includes two areas of circuits 702 sealed in cavities at each end of the intraocular lens 700. The intraocular lens 700 further includes a physiological sensor 704. As illustrated in FIG. 5, the pupil is partially constricted, leaving open an area 706 through which light passes, which may be detected by physiological sensor 704.

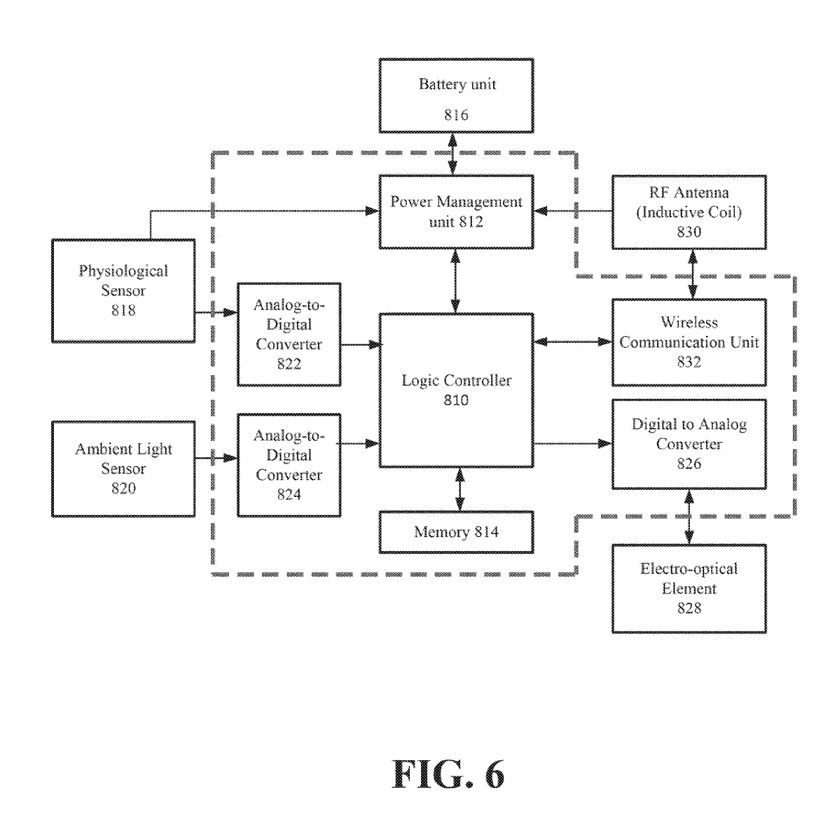

FIG. 6 provides an overview of some functional blocks in an embodiment of an intraocular lens. The dotted line around some of the functional blocks indicates implementation within a designated electronics area such as the area for the electronic circuits 702 of FIG. 5, whereas functional blocks outside the dotted line may be partially or wholly physically implemented in the designated electronics area or elsewhere on the intraocular lens. The functional blocks may be implemented as electronic circuits, which may include passive and active components, logic components, and components such as a processing device that executes instructions stored in a memory. Thus, a functional block represents some combination of hardware and software (including firmware). Each of the functional blocks shown may include sub-functions not shown, such as filtering, level shifting, or protection. The functional blocks in the embodiment depicted in FIG. 6 include a physiological sensor 818 for detecting a physiological response associated with ocular accommodation, an optional ambient light sensor 820 for detecting the ambient light level, analog-to-digital converters 822 and 824 for converting the analog sensor signals to digital signals, and a logic controller 810. The logic controller 810 receives and analyzes the digitized signals from the analog-to-digital converters 822 and 824 (i.e., the digitized versions of the signals from the physiological sensor 818 and the ambient light sensor 820, respectively).

The logic controller 810 executes instructions stored in a memory 814 to determine an appropriate adjustment of optical power for an electro-optical element 828 based on the analysis of the digitized signals from the analog-to-digital converters 822 and 824. The memory 814 may also store customized data, such as the pupil dynamics of a particular person (e.g., a characterization, calibrated characterization, or characterization with separate calibration parameters), for use by the logic controller 810 to determine the appropriate optical power adjustment for that particular person. The memory 814 may be any non-transitory computer-readable storage medium capable of storing or encoding a sequence of instructions or computer codes for performing the operations, methodologies, and techniques described herein. In some embodiments, the media and computer codes may be specially designed and constructed for the purposes of the disclosure. In other embodiments, the media and computer codes may be of a kind well known and available to those having skill in the art.

Based on the determined optical power adjustment and the electro-optical properties of the electro-optical element 828, the logic controller 810 can determine an appropriate control voltage or current to apply to the electro-optical element 828, and provide a corresponding signal to a digital-to-analog converter 826 such that the appropriate control voltage or current is applied. Alternatively, the logic controller 810 may directly determine the control voltage or current to apply to the electro-optical element 828, as described above, from the digitized signals. The logic controller 810 may communicate externally via a wireless communication unit 832; for example, to provide historical data or self-test data, or for reprogramming.

Power is provided to the functional blocks of FIG. 6 by way of a biocompatible battery in battery unit 816. The battery may be, for example, a solid-state ceramic power cell or a solid-state thin-film battery. The battery may have a long cycle life, such as more than about 8,000 cycles with more than about 1,000 hours of operation before a 100% discharge in each cycle. The battery further may have very low self-discharge, such as less than about 10% per year, less than about 5% per year, less than about 2% per year, or less than about 1% per year. In some embodiments, the battery can provide a total electrical energy in the range of, for example, about 30 to about 300 micro ampere-hours, or about 50 to about 200 micro ampere-hours. In some embodiments, the battery is flexible and can be folded or twisted without damage. The battery can be manufactured in different shapes and sizes. In some embodiments, the battery is an ultrathin power cell with a thickness in a stacked configuration such as, for example, less than about 1 mm, less than about 0.5 mm, or less than about 0.25 mm. In some embodiments, the battery unit 816 includes a rechargeable battery.

FIG. 6 further includes a power management unit 812 functional block. The power management unit 812 controls the distribution of power from the battery to other components of the electronic circuits. Power management unit 812 may further control the charging of a rechargeable battery in battery unit 816. Electrical power for charging the rechargeable battery may be received from the physiological sensor 818 and/or the ambient light sensor 820, one or both of which may be photovoltaic cells. Alternatively or additionally, the power for charging the rechargeable battery can be received via an RF antenna 830, which can be an inductive coil. In some embodiments, the RF antenna 830 includes a coil having about fifteen windings arranged around a perimeter of about 5.7 mm×2.6 mm.

In some embodiments, RF antenna 830 may also be used for wireless data communication with external devices, for example, to customize or reprogram the intraocular lens, or to update or extract information stored in a memory (e.g., memory 814). The operating band of the RF antenna may be, for example, in the Bluetooth, ZigBee, Wireless Fidelity (Wi-Fi), Worldwide Interoperability for Microwave Access (WiMAX), or Ultra-Wideband (UWB) operating bands. In some embodiments, the operating band of the RF antenna is in a near field communication (NFC) band or an inductive power transfer standard band, such as Qi. The wireless charging and data communication avoids the use of exposed contact leads for charging the battery and allows the hermetic sealing against safety hazards and performance degradation caused by fluid ingression.

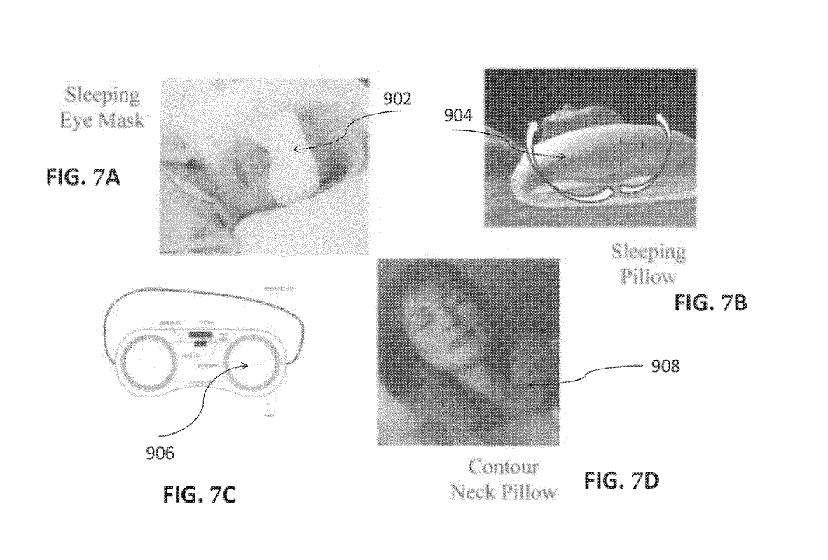

FIGS. 7A-7D illustrate different ways to wirelessly recharge a rechargeable battery in an intraocular lens, such as the rechargeable battery 816 in FIG. 6, through the RF antenna 830. External devices, such as an eye mask 902, a sleeping pillow 904, a goggles 906 and a neck pillow 908 may include a wireless charging mechanism.

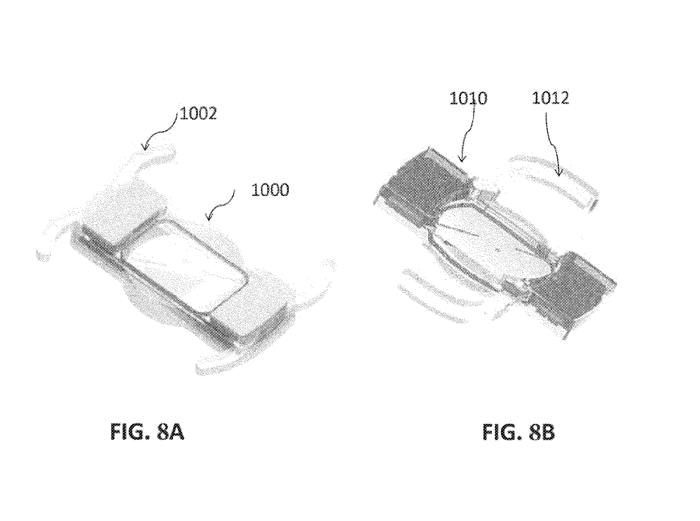

FIGS. 8A and 8B illustrate some embodiments of haptics in an intraocular lens. The performance of an intraocular lens design is highly dependent on the position of the intraocular lens in the optical system of an eye. The haptic design aids in the alignment and support of the intraocular lens within the eye. A haptic of the intraocular lens can be a plate haptic or open-loop. The haptic can be multi-piece or single-piece. FIG. 8A illustrates intraocular lens 1000, where two open-loop arms 1002 emanate from each edge of the lens 1000. FIG. 8B illustrates intraocular lens 1010, where one arm 1012 emanates from each side of the intraocular lens 1010. Additional arms may be used for either of the embodiments of FIGS. 8A or 8B. The arms hold the intraocular lens in place in the eye by exerting a centripetal pressure on vitreous chamber of the eye.

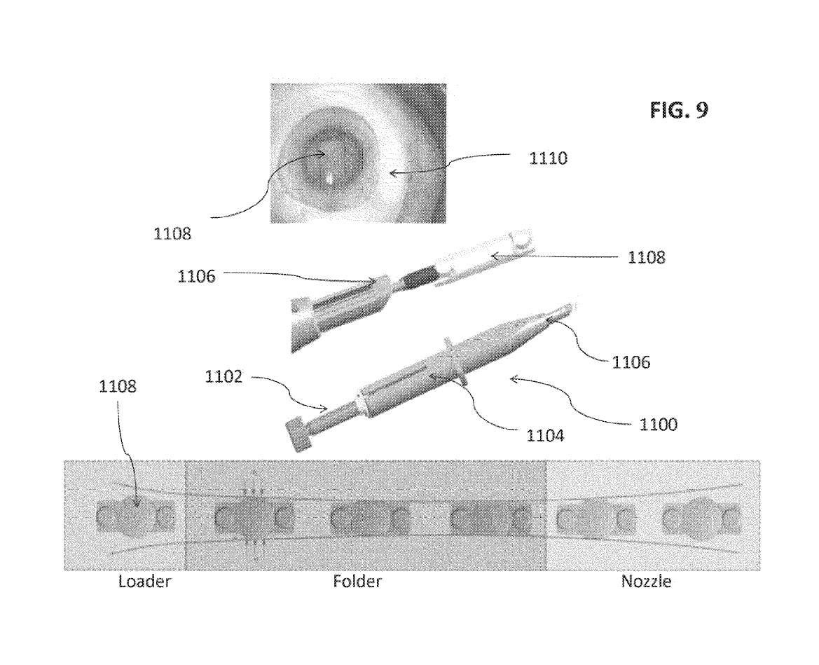

FIG. 9 illustrates a procedure for inserting an electro-optical intraocular lens 1108 into an eye 1110 of a patient. In some embodiments, the electro-optical intraocular lens 1108 can be implanted in the eye 1110 using an inserter 1100. In some embodiments, the inserter 1100 can include a loader section 1102 for loading the intraocular lens 1108 into the inserter 1100, a folder section 1104 that can compress the intraocular lens 1108 when the intraocular lens 1108 is being pushed through the folder section 1104 section by an injection force, and a nozzle section 1106 for release of the intraocular lens 1108 into the eye 1110.

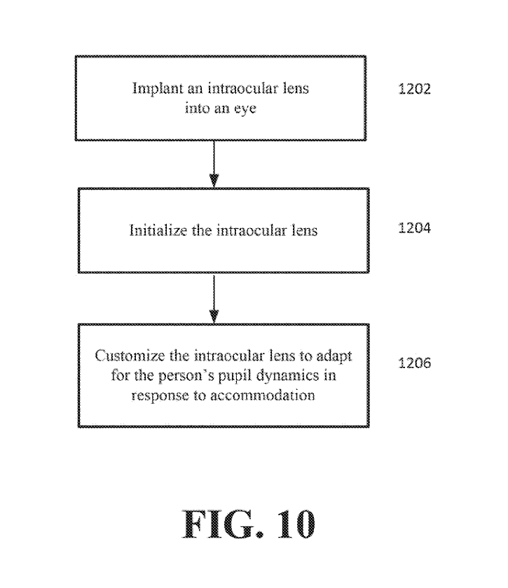

FIG. 10 illustrates the initialization and customization of an implanted electro-optical monofocal intraocular lens. After the electro-optical monofocal intraocular lens is implanted into the eye of a patient (block 1202), the lens can be initialized by executing an initialization program stored in the electronic circuits of the lens (block 1204). The initialization process in block 1204 can activate the components of the lens, and perform a diagnostic procedure to ensure that the lens is implanted properly and functions as expected. In some embodiments, instructions (i.e., stored in electronic circuits of an intraocular lens) can be executed to characterize or calibrate appropriate optical power adjustment for sight correction at different focal distances for the specific eye in which the lens is implanted (block 1206). After the electro-optical intraocular lens is properly implanted, initialized and customized, the intraocular lens can mimic or replace the eye's natural accommodation response by automatically adjusting the optical power of the intraocular lens based on the distance of the eye's focal target.

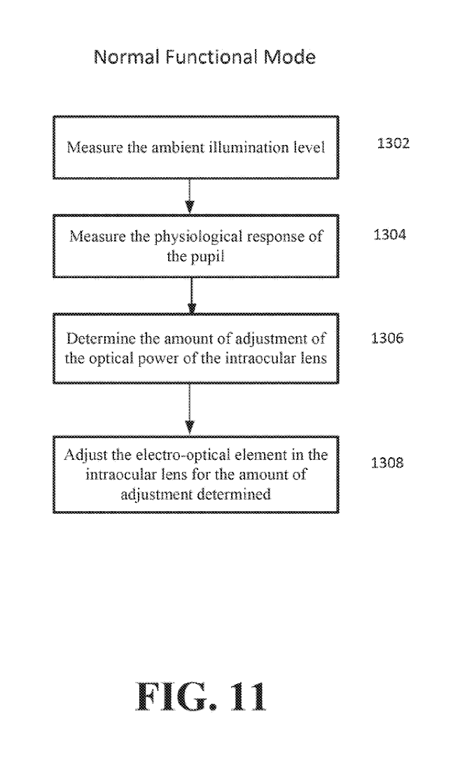

FIG. 11 illustrates for some embodiments the operations of an electro-optical intraocular lens in a normal function mode. The electro-optical element in an intraocular lens may be set to a default manual switching mode during initialization, and a physician can activate the normal functional mode after the implantation. In embodiments where the electro-optical intraocular lens includes a photosensor for detecting the ambient illumination level, the detected ambient illumination level is measured (block 1302). A physiological sensor in the intraocular lens measures the physiological response of an eye by detecting the illumination level or the change in illumination level on the plane where the intraocular lens is implanted (block 1304). The electronic circuits of the electro-optical intraocular lens can analyze the detected physiological response and the measured ambient illumination level to determine whether the eye changes its focal target from an object at one distance to another object at a different distance. Based on the determination, an appropriate optical power is identified (block 1306). The electronic circuits apply an appropriate control voltage or current to the electro-optical element (block 1308) to modify the optical power of the electro-optical element.

Pupil dynamics of a person may be noisy, meaning that the pupil diameter does not change in a smooth fashion, or does not stay steady even when the focal point does not change. In some embodiments, techniques may be included in the analysis to filter out the noise.

In some embodiments, it may be advantageous for the electro-optical element to have minimum optical power when no control voltage or current is applied. For example, if it is determined that the eye is trying to focus on a target at a long distance, the electronic circuits in the intraocular lens may not apply any control voltage or current (e.g., apply zero Volts or zero Amps, respectively) on the electro-optical element such that the optical power of the electro-optical element is at its default value of zero; and if it is determined that the eye is trying to focus on a target at a short distance, a control voltage or current is applied to achieve a particular optical power. Conversely, in some other embodiments, it may be advantageous for the electro-optical element to have maximum optical power when no control voltage or current is applied. In some embodiments, it may be advantageous for the electro-optical element to have maximum optical power at a predefined distance.

In some embodiments, the optical power of the electro-optical element in the intraocular lens can be continuously adjusted to accommodate for targets at different distances (where continuous adjustment is within the limits of the circuitry; for example, within the limits of resolution of an digital-to-analog converter or the control signal applied to the digital-to-analog converter). In some other embodiments, the electro-optical element can have multiple states, where the control voltage or current is set to discrete levels, and the depth of focus of the electro-optical element can provide functional vision for targets at focal distances within a range. In some embodiments, the electro-optical element can be a dual-state switchable device, the optical power of which is at one value, such as for distance view, when no control voltage or current is applied, and at another value, such as for near view, when a control voltage or current within a range is applied.

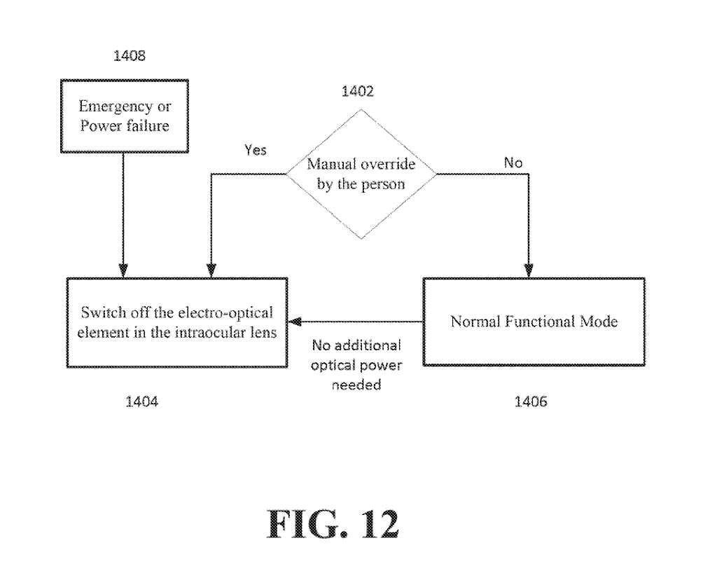

FIG. 12 illustrates some additional operations of the intraocular lens for some embodiments. The electro-optical element in the intraocular lens can be manually overridden (block 1402) to turn the electro-optical element off (block 1404). For example, the electro-optical element can be turned off by a remote controller, or by a predefined movement of the eye such as a specific blink sequence.

In some embodiments, the electro-optical element can be switched off in emergency situations through an emergency manually operated switch, or automatically when there is a power failure in the intraocular lens (block 1408). In some embodiments, in the normal functional mode (block 1406), the electro-optical element can also be switched off if it is determined that no additional optical power is needed at the present time.

While the concepts of this disclosure have been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt to a particular situation, material, composition of matter, technique, or operation and remain within the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure.

Accordingly, unless specifically indicated herein, the order and grouping of the operations is not a limitation of the disclosure.

While certain conditions and criteria are specified herein, it should be understood that these conditions and criteria apply to some embodiments of the disclosure, and that these conditions and criteria may not apply, or can be relaxed or otherwise modified. for other embodiments of the disclosure.

As used herein, the singular terms “a,” “an,” and “the” include plural referents unless the context clearly dictates otherwise. Thus, for example, reference to an object can include multiple objects unless the context clearly dictates otherwise.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, the terms can refer to less than or equal to ±5%, such as less than or equal to ±4%, less than or equal to ±3%, less than or equal to ±2%, less than or equal to ±1%, less than or equal to ±0.5%, less than or equal to ±0.1%, or less than or equal to ±0.05%

Those of skill would further appreciate that the various illustrative logical blocks, modules, circuits, and functional blocks described in connection with the disclosure herein may be implemented as electronic hardware, firmware, software, or combinations of hardware, firmware and software. Accordingly, various illustrative components, blocks, modules, circuits, and steps have been described above generally in terms of their functionality. Whether such functionality is implemented as hardware, firmware or software depends upon the particular application and design constraints imposed on the overall system. Skilled artisans may implement the described functionality in varying ways for each particular application, but such implementation decisions should not be interpreted as causing a departure from the scope of the present disclosure.

Certain aspects of the various illustrative logical blocks, modules, and circuits described in connection with the disclosure herein may be implemented or performed with a form of processor, such as a microcontroller, a microprocessor, a digital signal processor (DSP), an application specific integrated circuit (ASIC), a field programmable gate array (FPGA) or other programmable logic device, or a state machine. Certain aspects of the various illustrative logical blocks, modules, and circuits described in connection with the disclosure herein may be implemented or performed with discrete gate or transistor logic, discrete hardware components, or any combination thereof A processor may also be implemented as a combination of computing devices, e.g., a combination of a DSP and a microprocessor, a plurality of microprocessors, one or more microprocessors in conjunction with a DSP core, or any other such configuration.

Certain aspects of the various illustrative logical blocks, modules, and circuits described in connection with the disclosure herein may be implemented or performed using software. Software includes instructions stored on a computer-readable medium. By way of example, and not limitation, such computer-readable media can comprise RAM, ROM, or EEPROM.

Examples of software include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

What is claimed is:

1. An electronic intraocular lens, comprising:
a physiological sensor for detecting a physiological response of an eye associated with an ocular accommodation; and
an electro-optical element configured to adjust optical power based on the detected physiological response of the eye.

2. The electronic intraocular lens of claim 1, wherein the physiological sensor is configured to measure the illumination level at a plane where the electronic intraocular lens is implanted.

3. The electronic intraocular lens of claim 2, wherein the physiological sensor is a photovoltaic cell.

4. The electronic intraocular lens of claim 1, wherein the electro-optical element is a lens comprising transparent electrodes, and an electro-optical material filled in a cavity formed by a transparent material.

5. The electronic intraocular lens of claim 4, wherein the lens is one of a diffractive lens and a refractive lens.

6. The electronic intraocular lens of claim 1, further comprising a logic controller electrically connected to the physiological sensor and the electro-optical element, wherein the logic controller is configured to analyze the physiological response measured by the physiological sensor, determine an appropriate adjustment of the optical power based on the measured physiological response, and control the electro-optical element to achieve the appropriate adjustment of the optical power.

7. The electronic intraocular lens of claim 6, further comprising at least one non-volatile memory electrically connected to the logic controller, wherein the non-volatile memory stores codes to be executed by the logic controller to configure and control the electronic intraocular lens.

8. The electronic intraocular lens of claim 6, further comprising an ambient light sensor configured to measure the ambient light level, wherein the logic controller analyzes the measured physiological response together with the measured ambient light level to determine the appropriate adjustment of the optical power.

9. The electronic intraocular lens of claim 1, further comprising a rechargeable battery, and an antenna including an inductive coil for receiving energy to recharge the battery.

10. The electronic intraocular lens of claim 9, wherein the antenna is configured for wireless data communication in addition to wirelessly charging the rechargeable battery.

11. The electronic intraocular lens of claim 10, wherein the electronic intraocular lens can be remotely switched off through the antenna or manually switched off by a specific predefined movement of the eye.

12. The electronic intraocular lens of claim 1, further comprising:
at least one rechargeable battery; and
at least one of an antenna and a photovoltaic cell for charging the rechargeable battery.

13. The electronic intraocular lens of claim 12, wherein the rechargeable battery comprises a solid-state thin-film battery.

14. The electronic intraocular lens of claim 12, further comprising a power management circuit.

15. The electronic intraocular lens of claim 1, wherein the physiological sensor and the electro-optical element are encapsulated or hermetically sealed by at least one biocompatible material.

16. The electronic intraocular lens of claim 1, wherein the electro-optical element is capable of adjusting optical power continuously.

17. A monofocal intraocular lens, comprising:
a photovoltaic sensor for sensing pupil constriction and dilation;
an electronic circuit electrically connected to the photovoltaic sensor; and
an electrically adjustable optical element electrically connected to the electronic circuit;
wherein the electronic circuit analyzes a signal from the photovoltaic sensor and generates a control signal to cause a selected control voltage or current level to be applied to the electrically adjustable optical element, thereby modifying an optical power of the electrically switchable optical element.

18. The monofocal intraocular lens of claim 17, wherein the selected control voltage or current level switches the electrically adjustable optical element on or off, and the optical power of the electrically adjustable optical element is zero when switched off.

19. A method of artificial ocular accommodation, comprising:
detecting, by a physiological sensor, a physiological response of an eye of a person associated with a natural ocular accommodation; and
adjusting an optical power of an electro-optical element based on the detected physiological response of the eye.

20. The method of claim 19, further comprising at least one of
filtering noise of the detected physiological response; and
customizing the adjustment of the optical power of the electro-optical element based on the person's pupil dynamics.
